(19) United States
(12) Patent Application Publication
(10) Pub. No.: US 2026/0335592 A1
(43) Pub. Date: Oct. 29, 2026

(54) ENDOVASCULAR SYSTEM INCLUDING A VARIABLE STIFFNESS CATHETER WITH STEERABILITY

(71) Applicant: Daniel Ezra Walzman, Teaneck, NJ (US)

(72) Inventor: Daniel Ezra Walzman, Teaneck, NJ (US)

(21) Appl. No.: 19/107,915

(22) PCT Filed: Sep. 9, 2024
(86) PCT No.: PCT/US2024/045764

§ 371 Date: Feb. 28, 2025

Related U.S. Application Data
(60) Provisional application No. 63/538,095, filed on Sep. 13, 2023.
(60) Provisional application No. 63/690,364, filed on Sep. 4, 2024.
(60) Provisional application No. 63/691,442, filed on Sep. 6, 2024.

(51) Int. Cl. A61M 25/00; A61M 25/01

(52) U.S. Cl. CPC A61M 25/0053; A61M 25/0068; A61M 25/0147; A61M 2205/0266; A61M 2210/12

(57) ABSTRACT

System including an elongated member and a steering assembly that is connected to the elongate member, and which is configured to facilitate reconfiguration thereof. The elongated member includes a first segment and a second segment that is located distally of the first segment, wherein the second segment includes a stiffness that can be increased or decreased to reconfigure the elongated member via movement of a stiffening member.

### BACKGROUND

This application claims priority from provisional application Ser. No. 63/538,095, filed Sep. 13, 2023, provisional application Ser. No. 63/690,364, filed Sep. 4, 2024, and provisional application Ser. No. 63/691,442, filed Sep. 6, 2024. The entire contents of each of these applications are incorporated herein by reference.

### FIELD OF THE INVENTION

The present invention relates to catheter based systems and methods, and more particularly, to a steerable catheter that includes a variable stiffness in order to allow for controlled bending of the catheter during a procedure.

### BACKGROUND OF THE INVENTION

Access to a patient's blood vessels is necessary for a wide variety of medical, diagnostic, and therapeutic purposes, which often includes navigating a long and tortuous path.

While a wide variety of catheters have been developed in order to improve vascular access, a need remains for a catheter that offers greater precision and control during placement and use. Steerable catheters are known. In these steerable catheters, the length of the curve zone and their bend radius is fixed by their design as upon actuating of steering mechanisms, such as cables or wires, the catheter will bend at a predetermined region, in a predetermined shape, and with a predetermined radius of curvature.

It would be beneficial to provide steerable catheters with improved versatility which would not only facilitate surgical procedures but provide opportunities for use in a larger range/variety of procedures.

### SUMMARY OF THE INVENTION

The present invention provides a steerable catheter that overcomes the problems and deficiencies of the prior art. In general, the present invention provides a catheter with an adjustable stiffness which is utilized to adjust the length of the curve zone and the bend radius of the catheter. Various embodiments of the catheter of the present invention are discussed in detail below. The catheter in some embodiments can include an integrated video camera.

In one aspect of the present invention, a system, and in some embodiments an endovascular system, is provided that is configured for use during surgical procedures such as an endovascular procedure. The system includes: a catheter that is configured in one embodiment for insertion into a patient's blood vessel; an articulation (steering) assembly that is connected to the catheter and which is configured to facilitate reconfiguration thereof; and a stiffening assembly. The catheter includes: a proximal segment and at least one steerable segment that is located distal to the proximal segment, wherein the proximal segment has a first stiffness, and the steerable segment has a second stiffness less than the first stiffness to facilitate bending of the steerable segment during reconfiguration of the catheter, and to prevent bending of the proximal segment. The stiffening assembly is configured for movement with respect to the steerable segment to alter (e.g., increase or decrease) the second stiffness and adjust (e.g., restrict) bending thereof.

In some embodiments, the stiffening assembly may be configured for movement within the steerable segment to increase the stiffness of a section of the steerable segment and restrict bending thereof. Such movement can be internal or external with respect to the steerable segment.

In some embodiments, the articulation (steering) assembly may include at least one articulation (steering member), and a tensioning mechanism. The at least one articulation member extends within the catheter and is secured thereto, and the tensioning mechanism is connected to the at least one articulation member such that, upon actuation of the tensioning mechanism, a force is applied to the catheter via the at least one articulation member to facilitate reconfiguration thereof.

The tensioning mechanism in the embodiments disclosed herein can include one or both of a push mechanism (moving distally for catheter bending) or a pull mechanism (moving proximally for catheter bending).

In preferred embodiments the catheter has a continuous and smooth outer surface, without separate nodes to articulate relative to each other.

In some embodiments, the at least one articulation member may extend substantially within an outer wall of the catheter.

In some embodiments, the at least one articulation member may extend within a channel that is defined by the outer wall of the catheter.

In some embodiments, the at least one articulation member may include a) first articulation member that is secured to the catheter such that upon actuation of the tensioning mechanism, the first articulation member causes deflection of the catheter in a first direction; and b) second articulation member that is secured to the catheter such that, upon actuation of the tensioning mechanism, the second articulation member causes deflection of the catheter in a second direction. In some embodiments, the second direction may be generally opposite to the first direction. Alternatively, it could be in the same or other directions.

In some embodiments, the catheter may be configured such that the second stiffness of the steerable segment is less than or equal to the first stiffness.

In some embodiments, the proximal segment may include a first material having a first durometer, and the steerable segment may include a second material having a second durometer that is less than the first durometer.

In some embodiments, the first segment and second segment have different materials to create different stiffnesses. In some embodiments, the first segment and second segment have different layers to create different stiffnesses.

In some embodiments, the stiffening assembly may include a push member (e.g., an elongated member such as a push rod, a push wire, a hypotube, etc.), and a stiffening member that is secured to the push assembly such that movement of the push assembly causes corresponding movement of the stiffening member. In other embodiments, the stiffening assembly can include a pull assembly pulled to move the stiffening member.

In some embodiments the stiffening assembly can be pulled and/or pushed.

In some embodiments, the stiffening assembly may include more than one member, which may move together or separately and may extend along an identical length of the catheter, may extend along distinct (e.g., different) lengths of the catheter, and/or may partially overlap.

In some embodiments, the endovascular system may further include a controller that is connected to the push assembly, and which is configured to cause axial movement of the stiffening assembly within the steerable segment, and thereby adjust the length of the steerable segment.

In some embodiments, the articulation assembly may be connected to the controller.

In some embodiments, the steerable segment may include at least one shape memory material such that, upon exposure to an external stimulus, or removal of an external restraint, the steerable segment assumes a predetermined configuration.

In another aspect of the present invention, a system is provided that is configured for use during a procedure. The system includes an elongated member and an articulation (steering) assembly that is connected to the elongated member, and which is configured to facilitate reconfiguration thereof. The elongated member includes: a first segment and a second segment that is located distally of the first segment, wherein the second segment includes a variable stiffness that can be altered, i.e., increased or decreased, to influence reconfiguration of the elongated member. The stiffness is increased or decreased to increase or decrease bending to influence a radius of the curve of the second segment.

In some embodiments, the second segment may include at least one shape memory material. In some embodiments, the at least one shape memory material may be responsive to an external stimulus such that, upon exposure to the external stimulus, or removal of an external restraint, the variable stiffness of the second segment is increased.

In some embodiments the shape memory material(s) may encourage the second segment to form a certain configuration (e.g., a bend having a particular shape) upon reconfiguration of the elongated member.

In some embodiments, different laser cut hypotubes can be used. The hypotubes can have different cut patterns. The hypotubes may be incorporated into the wall of the device. Hypotubes may be full circumference or partial circumference.

In some embodiments, the elongated member may include at least one chamber that is located within the second segment, and which is configured to receive a fluid (or other suitable substance) from a source such that upon communication of the fluid (substance) into the at least one chamber, the second segment varies (changes) (i.e., increases or decreases) in stiffness.

In some embodiments, the at least one chamber may include a plurality of chambers, which may be along the same length of the elongated member, may be along different lengths of the elongated member, and/or may partially overlap. In such embodiments, the fluid (substance) may be delivered to and/or removed from one or more of the plurality of chambers to further increase control over the stiffness of the second segment.

In some embodiments, the first segment may include a first material having a first durometer, and the second segment may include a second material having a second durometer that is less than the first durometer such that the second segment includes a stiffness that is less than that of the first segment. Variations of stiffness within segments may further influence bending and straightening, as well as shapes.

In some embodiments, the elongated member is a catheter; in other embodiments, the elongated member is an endoscope.

In some embodiments, the system may further include at least one stiffening assembly that is axially movable within the second segment to increase and decrease the stiffness thereof.

In another aspect of the present invention, a method of performing a procedure, such as an endovascular procedure, is provided. The method includes: a) inserting a catheter (or endoscope) into a blood vessel or luminal organ in a first configuration, wherein the catheter includes: a first segment and a second segment that is located distally of the first segment; b) advancing the catheter towards a target site; c) actuating an articulation (steering) assembly that is connected to the catheter to thereby reconfigure the catheter from the first configuration into a second configuration via bending of the second segment; and d) either before or after step (c), increasing a stiffness of the second segment to alter (e.g., reduce) bending thereof.

In some embodiments, increasing the stiffness of the second segment may include advancing a stiffening assembly axially within the second segment (e.g., within a region of the second segment, internally or externally thereof).

In some embodiments, increasing the stiffness of the second segment may include exposing the second segment to an external stimulus.

In some embodiments, increasing the stiffness of the second segment may include communicating a fluid (or other suitable substance) into at least one chamber that is located within the second segment.

### BRIEF DESCRIPTION OF THE DRAWINGS

FIG. 1 is a schematic view of an endovascular system according to the principles of the present invention, which includes: a catheter; an articulation (steering) assembly; and a stiffening assembly.

FIG. 2 is a transverse (horizontal) cross-sectional view taken along line 2 -2 in FIG. 1.

FIG. 3 is a schematic view of an alternate embodiment of the catheter of FIG. 1, which includes a side hole.

FIG. 4 is a schematic view of an alternate embodiment of the endovascular system of FIG. 1, in which the articulation assembly includes a pair of articulation (steering) members.

FIG. 5 is a transverse (horizontal) cross-sectional view taken along line 5 -5 in FIG. 4.

FIG. 6 is a schematic view of an alternate embodiment of the endovascular system, in which the catheter includes a retention member (ring) that is secured (connected) to the articulation assembly.

FIG. 7 is a transverse (horizontal) cross-sectional view taken along line 7 -7 in FIG. 6.

FIG. 8 is a schematic view of an alternate embodiment of the endovascular system, which includes a controller.

FIG. 9A is a schematic view of the catheter of FIG. 1, which is shown in a deflected configuration and exhibiting a curvature of (approximately) 90 degrees.

FIG. 9B is a schematic view of the catheter of FIG. 1, which is shown in a deflected configuration and exhibiting a curvature of (approximately) 180 degrees with a first radius of curvature.

FIG. 9C is a schematic view of the catheter of FIG. 1, which is shown in a deflected configuration and exhibiting a curvature of (approximately) 180 degrees with a second, reduced radius of curvature.

FIG. 10 is a schematic view of the catheter of FIG. 1, which is shown in the deflected configuration with the steering assembly advanced to a first (axial) location.

FIG. 11A is a schematic view of the catheter of FIG. 1, which is shown in the deflected configuration with the steering assembly advanced to a second (axial) location.

FIG. 11B is a schematic view of the catheter of FIG. 1, which is shown in the deflected configuration with the steering assembly advanced to a third (axial) location.

FIG. 12 is a schematic view of the catheter of FIG. 1 shown with an alternate embodiment of the stiffening assembly.

FIG. 13 is a side, perspective view of the stiffening assembly of FIG. 12.

FIG. 14 is a schematic view of an alternate embodiment of the catheter of FIG. 1, which includes (one or more) at least one shape memory material.

FIG. 15 is a schematic view of an alternate embodiment of the endovascular system, in which the catheter includes (one or more) at least one (internal) chamber that is in (fluid) communication with a source.

FIG. 16 is a transverse (horizontal) cross-sectional view taken along line 16-16.

### DETAILED DESCRIPTION OF THE INVENTION

The present invention provides a system that is configured for use during a procedure in which one or more blood vessels or luminal organs or similar structures are accessed and treated (e.g., to remove a blockage, administer a therapeutic compound, biopsy, etc.). The system, e.g., endovascular system includes a variable stiffness, steerable (articulable) catheter that is configured for insertion into a patient's blood vessel (or other body regions), and an articulation (steering) assembly that is secured (connected) to the catheter, and which is configured to facilitate steering (articulation) thereof via reconfiguration (e.g., bending) of the catheter. The catheter includes: a proximal segment; a distal segment; and at least one steerable segment that is located between the proximal segment and the distal segment. Multiple steerable segments, optionally separated by non-steerable segments of various length, and/or partially or fully overlapping, are envisioned as well. The stiffness of the steerable segment (in whole or in part) can be increased or decreased during use of the system, i.e., during the course of the surgical procedure, e.g., prior to or after insertion into the body, which allows for greater precision and control during the placement and orientation thereof as it allows for changing the length of the “curve zone” and altering the radius and/or shape bend.

In one embodiment, the system further includes a stiffening assembly that is movable within the steerable segment in order to vary the stiffness thereof and, thus, facilitate or restrict bending of the steerable segment. In such embodiments, in order to reduce the stiffness of the steerable segment (e.g., in relation to the proximal segment) and, thus, facilitate bending during reconfiguration of the catheter, it is envisioned that the catheter may include an outer wall with a reduced radial thickness in the steerable segment. Additionally, or alternatively, it is envisioned that the steerable segment may include a lower durometer than the proximal segment (e.g., via the incorporation of different materials of construction).

In another embodiment, the variable stiffness in the steerable segment is achieved through the incorporation of (one or more) at least one shape memory material that is responsive to an external stimulus such that, upon exposure to the external stimulus, the stiffness of the steerable segment is increased or decreased. It is envisioned that the shape memory material(s) may allow the steerable segment to form a certain configuration (e.g., a bend having a particular shape). In such embodiments, a mechanism may be incorporated to adjust the length of the steerable segment and thereby adjust the radius of curvature of the bend. In some embodiments, at least one hypotube with various cut patterns incorporated into the device wall may be used to facilitate bend(s) of particular radius and/or shape.

In some embodiments, the shape memory material(s) may encourage the second segment to form a certain configuration (e.g., a bend having a particular shape) upon reconfiguration of the catheter.

In another embodiment, the variable stiffness in the steerable segment is achieved through the inclusion of (one or more) at least one pocket or chamber that is located within the steerable segment, and which is configured to receive a fluid (substance) from a source such that, upon communication of the fluid (substance) into and out of the at least one pocket, the stiffness of the steerable segment is increased and decreased, respectively.

In some embodiments described herein with a movable stiffening member, preferably situated substantially within the wall of the device, by movement of the stiffening member of the catheter distally into the curve zone, it increases the resistance to bending at that region, thus decreasing the length of the curve zone and creating a tighter radius bend. If the stiffening member is moved proximally, it will increase the length of the curve zone creating a greater radius bend. Various forms of a stiffening member can be provided such as for example, one or more wires, one or more hypotubes of full or partial circumference, etc. The stiffening element is preferably attached on at least its proximal end to at least one connector, such as a wire or post, that can be adjusted by pulling or pushing the connector to adjust the position of the stiffening member. The connector preferably attaches to a mechanism on a handle near one end of the device, that further incorporates a mechanism to move the connector.

Although the systems and methods described herein are generally discussed in the context of a catheter, the principles of the present disclosure may be applied to a scope, instrument, or other such medical device.

As used herein, the term “proximal” refers to the section, portion, component, etc. closer to the user and the term “distal” refers to the section, portion, component, etc. further from the user. Ther terms “articulation” and “steering” are used interchangeably herein.

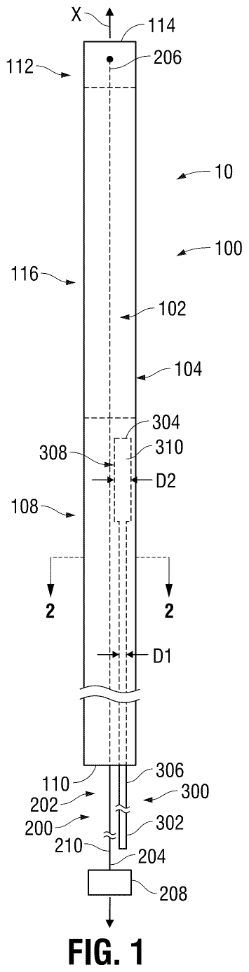

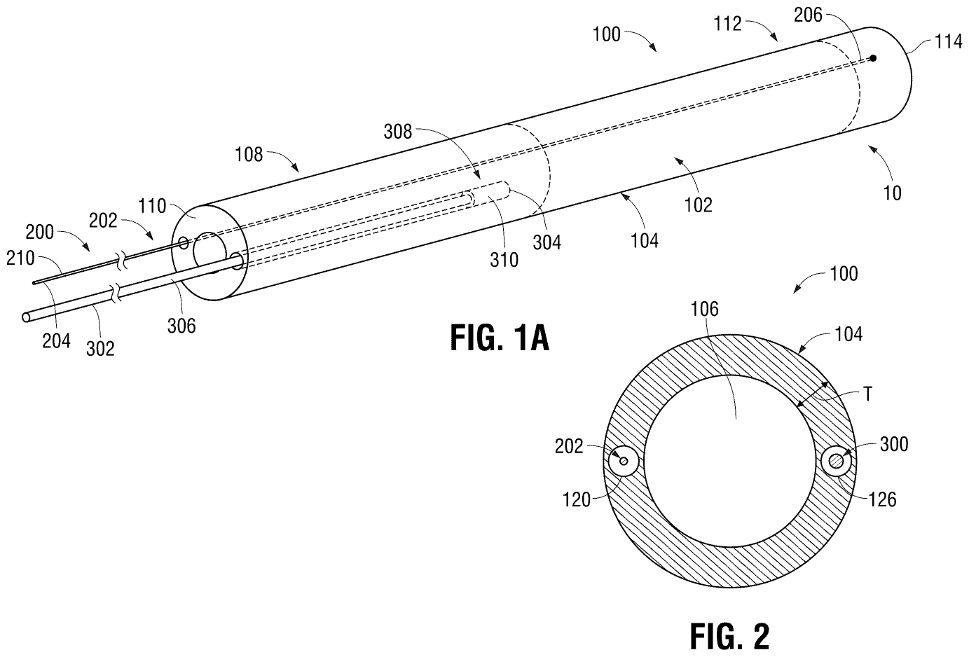

Turning now to the drawings wherein like reference numerals identify similar structural features of the devices and systems disclosed herein, and with initial reference to FIG. 1, an endovascular system 10 is illustrated that is configured for use during an endovascular procedure. More specifically, the endovascular system 10 includes: a catheter (an elongated member) 100; an articulation (steering) assembly 200, which is operatively secured (operatively connected) to the catheter 100 and is configured to facilitate steering (articulation) thereof via reconfiguration (e.g., bending) of the catheter 100; and a stiffening assembly 300.

Note the term articulation assembly can also be referred to as a bending assembly or a steering assembly.

The catheter 100 is configured for insertion into a patient's blood vessel or other body space or body cavity, and may include any suitable material or combination of materials. For example, it is envisioned that the catheter 100 may include (one or more) at least one metallic material (e.g., stainless steel, titanium, etc.) or (one or more) at least one non-metallic material (e.g., plastic material(s), polymeric material(s), composite material(s), etc.), either exclusively or in combination. Coils and/or braids and/or hypotubes with optional cut patterns may optionally be included along the entire length of the catheter 100 or along a portion of the length of the catheter 100.

The catheter 100 includes a body 102 that defines a longitudinal axis X. More specifically, the body 102 includes: an outer wall 104, which defines a main (working) lumen 106 (FIG. 2) that extends in generally parallel relation to the longitudinal axis X; a proximal (first) segment (zone) 108, which defines a proximal end hole 110; a distal (second) segment (zone) 112, which defines a distal end hole 114; and a steerable (third, deflectable, intermediate) segment (zone) 116, which is located between the respective proximal and distal segments 108, 112 such that the steerable segment 116 is located distally of the proximal segment 108, and the distal segment 112 is located distally of the steerable segment 116 in this embodiment. Dotted lines in FIG. 1 (and other Figures) provide one example of the demarcation of the segments 112, 116 and 108. It should be appreciated that these demarcations provide one example as different lengths of the segments other than those illustrated are also contemplated. As discussed below, these various segments can be formed of a single catheter of different materials (different stiffnesses) in some embodiments or formed of separate catheter segments of different materials or stiffnesses secured together. Variations may exist along the length and/or the circumference, and/or combinations thereof.

Although generally shown and described as including the proximal segment 108, the distal segment 112, and the steerable segment 116, embodiments of the catheter 100 that are devoid of the distal segment 112 are also envisioned herein (e.g., embodiments in which the steerable segment 116 includes the distal end hole 114), however, and would not be beyond the scope of the present disclosure. The distal segment 112 is thus an optional component of the catheter 100 that may be omitted in certain embodiments. Additionally, there may be multiple steerable segments along various lengths of the catheter. These may be along overlapping, nonoverlapping, and/or partially overlapping lengths. Directions of bend may optionally vary as well.

While the main lumen 106 is illustrated as including a generally annular (i.e., circular) configuration, it should be appreciated that the specific configuration of the main lumen 106 and/or the outer configuration of the catheter body (i.e., configuration of the outer wall 104) may be altered in various embodiments of the catheter 100 without departing from the scope of the present disclosure, e.g., it can be oval, rectangular, etc.

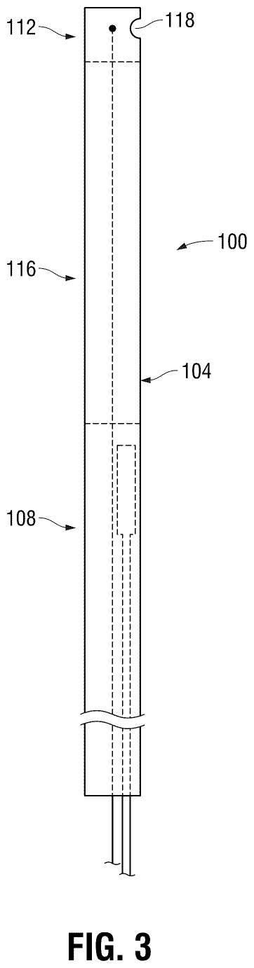

In some embodiments, such as that which is illustrated in FIG. 3, the catheter 100 may further include (one or more) at least one side hole 118 that extends through the outer wall 104 in order to provide access to the main lumen 106 (FIG. 2). For example, it is envisioned that the side hole(s) 118 may be configured to receive a surgical instrument (e.g., a second catheter), permit fluid flow therethrough, or facilitate any other therapeutic effect. Although shown as including a single side hole 118 that is located within the distal segment 112, it should be appreciated that the particular number, size and shape of side holes 118 and/or the location(s) thereof may be varied in alternate embodiments without departing from the scope of the present disclosure, and that the side hole(s) 118 may be incorporated in any and/or all segments of the catheter 100. For example, embodiments are envisioned in which the side hole 118 may be located within the proximal segment 108 or the steerable segment 116, as are embodiments in which the catheter 100 may include a plurality of side holes 118 that are located in one or more (at least one) of the proximal segment 108, the distal segment 112, and/or the steerable segment 116. In such embodiments, it is envisioned that the side holes 118 may be oriented in angular (circumferential) alignment so they are axially aligned, or that the side holes 118 may be angularly (circumferentially) offset from each other.

As described in further detail below, the stiffness of catheter 100 varies along the longitudinal axis X (FIG. 1) and is preferably non-uniform in rigidity, which improves access to target sites within the patient by increasing control over the configuration of the catheter 100 and, thus, more precise placement and orientation thereof. More specifically, the proximal segment 108 has a first stiffness, the steerable segment 116 has a second stiffness that is typically less than the first stiffness, which facilitates bending of the steerable segment 116 during reconfiguration of the catheter 100, and the distal segment 112 has a third stiffness, which is preferably greater than the second stiffness. Embodiments in which the third stiffness of the catheters of the various embodiments disclosed herein may be less than or equal to the second stiffness and/or greater or less than the first stiffness are also envisioned herein. Within each segment, it is envisioned that there may optionally be regional variations in stiffness as well.

In some embodiments, the catheter 100 may be unitary (e.g., monolithic) in construction such that the proximal segment 108, the distal segment 112, and the steerable segment 116 each include (e.g., are formed from) the same material. In such embodiments, the stiffness of the catheter 100 may be varied by reducing a radial thickness T (FIG. 2) of the outer wall 104 in one or more locations. For example, the catheter 100 may be configured such that the outer wall 104 defines a (first) radial thickness in the proximal segment 108, a (second) radial thickness in the steerable segment 116 that is less than the radial thickness in the proximal segment 108, and a (third) radial thickness in the distal segment 112, which may be less than or equal to the radial thickness in the proximal segment 108, but greater than the radial thickness in the steerable segment 116. Variations in braid pattern of an additional braid layer in the wall, and/or variation in cut patterns in additional hypotube(s) within the wall, and similar modalities to vary stiffness are envisioned as well.

Alternatively, the catheter 100 may include a multi-material construction such that the stiffness of the catheter 100 may be varied by incorporating different materials of construction in the proximal segment 108, the steerable segment 116, and/or the distal segment 112. For example, the catheter 100 may be configured such that the proximal segment 108 includes (e.g., is formed from) a first material having a first durometer, the steerable segment 116 includes (e.g., is formed from) a second material having a second durometer that is less than the first durometer, and the distal segment 112 includes (e.g., is formed from) either the first material or a third material having a third durometer, which may be less than or equal to the first durometer, but greater than the second durometer. In such embodiments, the proximal segment 108, the distal segment 112, and the steerable segment 116 may be formed as discrete components of the catheter 100 that can be secured (connected) together in any suitable manner (e.g., via an adhesive, via ultrasonic welding, heat welding, melting, laser welding etc.). Liners, coils, weaves, etc. and/or combinations of all of the above, and/or other components, could alternatively be utilized to form segments of different stiffnesses. It is also envisioned that the catheter 100 may also include one or more (optional) transition zones in which a more gradual change in stiffness occurs. It is envisioned that in some embodiments, the first segment and second segment have different materials to create different stiffnesses. It is also envisioned in some embodiments the first segment and second segment have different layers to create different stiffnesses.

The articulation assembly 200 is secured (connected) to the distal segment 112, and is configured to apply a force thereto in order to facilitate reconfiguration, i.e., bending, of the catheter 100. More specifically, upon actuation of the articulation assembly 200, the force applied to the catheter 100 (e.g., the distal segment 112) causes the steerable segment 116 to bend (curve, deflect), as described in further detail below, which is facilitated by the reduced stiffness thereof (e.g., in relation to the proximal segment 108). This bend region is referred to herein as the curve zone.

The articulation assembly 200 includes (one or more) at least one articulation (steering) member 202 having a proximal end 204 and a distal end 206, which is secured (connected) to the catheter 100, and (one or more) at least one tensioning mechanism 208, which is secured (connected) to the proximal end(s) 204 of the articulation member(s) 202. Although shown as being secured (connected) to the distal segment 112 in the illustrated embodiment, it is envisioned that the articulation member(s) 202 may be secured (connected) to the catheter 100 in any location suitable for the intended purpose of facilitating reconfiguration of the catheter 100 in the manner described herein. For example, an embodiment of the endovascular system 10 including (one or more) at least one articulation member 202 that is secured (connected) to the steerable segment 116 is also envisioned herein. In a preferred embodiment, the articulation member(s) are secured to a hypotube embedded in the catheter, preferably near the distal end of the curve zone. This hypotube is in some embodiments sometimes referred to as a pull ring, and may be of various shapes, circumferences, etc.

In some embodiments, the articulation member(s) 202 may be in-line with the catheter 100 and/or other devices. In other embodiments, the articulation member(s) 202 may be offset (e.g., located within a branched side location), which may be located within the proximal segment 108 (e.g., in proximate or adjacent relation to the proximal end hole 110).

In various embodiments of the disclosure, the articulation member(s) 202 may be either flexible or rigid in construction. For example, the articulation member(s) 202 may be configured as pull members, e.g., pull wire(s) 210 (FIG. 1), which enables the application of a proximally-directed pulling force to the catheter 100 (e.g., the distal segment 112). Alternatively, it is envisioned that the articulation member(s) 202 may be configured as push members, e.g., push rod(s), cable(s) and/or wire(s), which enables the application of a both a proximally-directed pulling force and a distally-directed pushing force to the catheter 100 (e.g., the distal segment 112). In some embodiments, it is envisioned that both push and pull forces may be applied to the same articulation member(s) 202, whereas in other embodiments, it is envisioned that the push and pull forces may be applied to different articulation members 202. At the proximal end the push/pull member(s) are optionally branched out of the wall of the catheter, and attach to a mechanism that is capable of moving it in a longitudinal direction along the catheter, via applying push and/or pull forces. The mechanism may incorporate at least one wheel and/or lever and/or similar mechanisms. These may be incorporated in-line with the catheter, and/or branched at an angle. A gear mechanism(s) may optionally be incorporated as well. Similar apparatuses and mechanisms may also be used to control stiffening member positioning as well.

In some embodiments, such as those illustrated throughout the figures, the articulation member(s) 202 may be spaced radially from the main lumen 106 and may extend substantially within the outer wall 104 of the catheter 100. More specifically, the articulation member(s) 202 extend within corresponding channel(s) 120 that are defined by the outer wall 104, and which preferably extend in generally parallel relation to the longitudinal axis X (FIG. 1) and the main lumen 106.

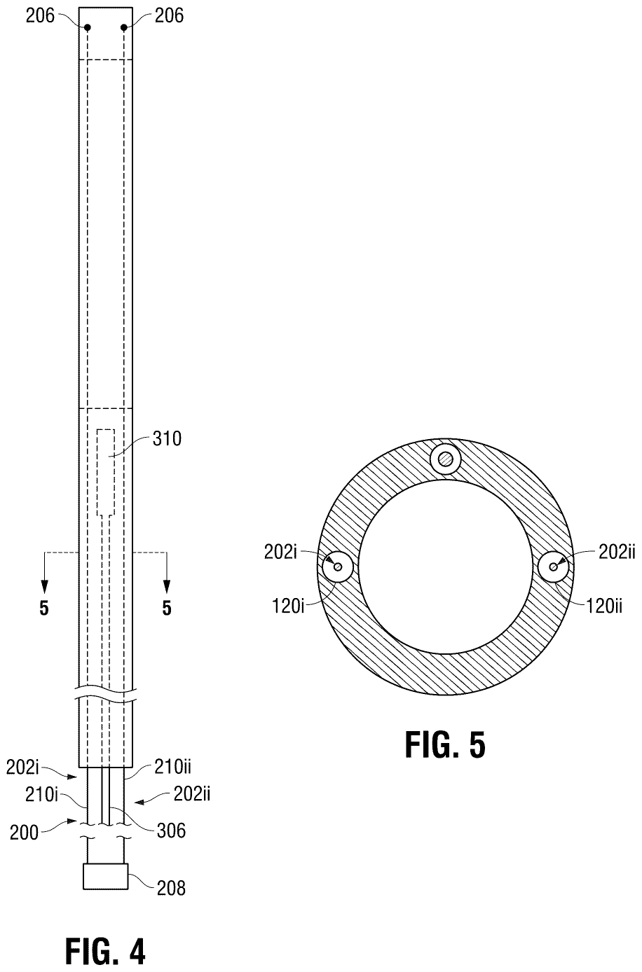

In the embodiment illustrated in FIGS. 1 and 2, the articulation assembly 200 includes a single articulation member 202 that is received within a corresponding channel 120, which allows for deflection of the catheter 100 (e.g., the distal segment 112) in a single direction only (e.g., when configured to exclusively apply a push force or a pull force). It should be appreciated, however, that the specific number of articulation members 202 and channels 120 may be increased in alternate embodiments in order to facilitate deflection of the catheter 100 (e.g., the distal segment 112) in multiple directions (e.g., when configured to apply a push force and a pull force so as to facilitate deflection (bending) in opposite directions). For example, FIGS. 4 and 5 illustrate an embodiment in which the articulation assembly 200 includes a pair of (e.g., first (primary) and second (secondary)) articulation members 202i, 202ii, which can be configured as pull wires, e.g., pull wires 210i, 210ii, that extend through corresponding (first and second) channels 120i, 120ii in the outer wall 104. More specifically, the (first) articulation member 202i is secured (connected) to the catheter 100 (e.g., the distal segment 112) such that, upon actuation of the tensioning mechanism(s) 208, the (first) articulation member 202i causes deflection of the catheter 100 in a first direction, and the (second) articulation member 202ii is secured (connected) to the catheter 100 (e.g., the distal segment 112) such that, upon actuation of the tensioning mechanism(s) 208, the (second) articulation member 202ii causes deflection of the catheter 100 in a second direction that is generally opposite to the first direction (e.g., when a pull force or a push force is applied to each of the articulation members 202 in the same direction). A single catheter alternatively may be configured in various different directions. Embodiments including three or more articulation members 202 and corresponding channels 120 are also envisioned herein.

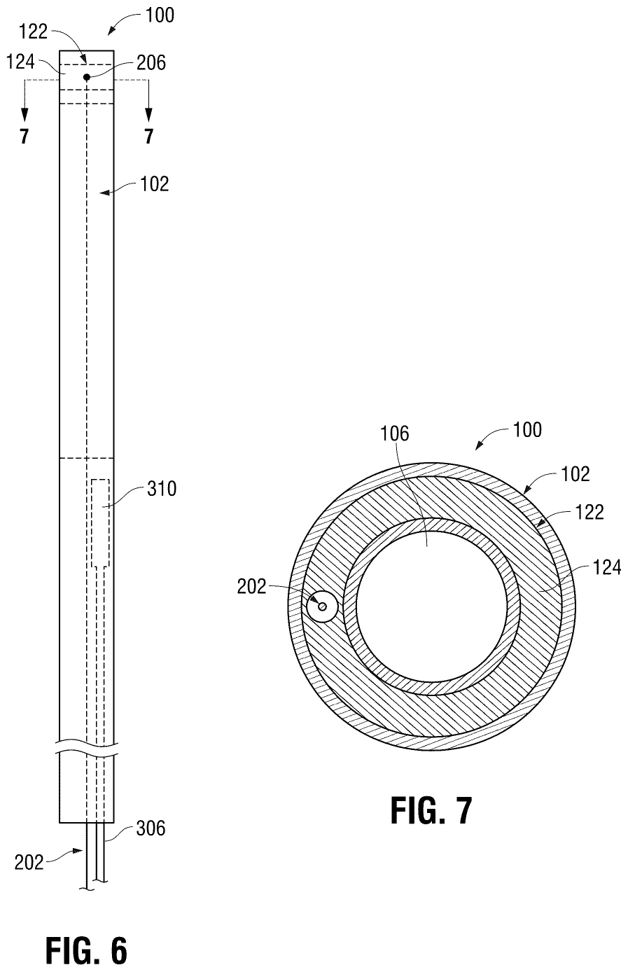

The distal end(s) 206 of the articulation member(s) 202 may be secured (connected) to the catheter 100 (e.g., the distal segment 112) in any suitable manner. For example, the distal end(s) 206 of the articulation member(s) 202 may be directly secured (connected) to the body 102 (e.g., within the channel(s) 120) such as, for example, via an adhesive, via ultrasonic welding, heat welding, laser welding, etc., as seen in FIG. 1. Alternatively, the distal end(s) 206 of the articulation member(s) 202 may be indirectly secured (connected) to the body 102. For example, in the embodiment illustrated in FIGS. 6 and 7, the catheter 100 includes a retention member 122 that is secured (connected) to the body 102 and the distal end(s) 206 of the articulation member(s) 202. Although shown as being configured as a generally annular retention ring 124 in FIGS. 6 and 7, it should be appreciated that the specific configuration of the retention member 122 may be varied in alternate embodiments. For example, an embodiment in which the retention member 122 may be crescent shaped (e.g., semicircular) in configuration is also envisioned herein, and would not be beyond the scope of the present disclosure. The retention member in one embodiment comprises a hypotube or other substantially cylindrical member substantially embedded in the wall of the catheter.

Although shown as including a single retention member 122 in FIGS. 6 and 7, it should be appreciated that the specific number of retention members 122 may be increased in alternate embodiments of the disclosure. Additionally, embodiments are envisioned in which the catheter 100 may include a plurality of retention members 122 that are associated with (e.g., secured (connected) to) multiple (distinct) steerable segments 116. Each retention member may also be connected to multiple independent articulation members.

The tensioning mechanism(s) 208 may include any device, structure, or the like suitable for the intended purpose of applying force to the proximal end(s) 204 of the articulation member(s) 202 such that, upon actuation of the tensioning mechanism(s) 208, force is applied to the catheter 100 (e.g., the distal segment 112) via the articulation member(s) 202 in order to facilitate bending of the steerable segment 116 and reconfiguration of the catheter 100. For example, the tensioning mechanism(s) 208 may include a rotating wheel, a pulley system, a ratchet, a lever, or the like.

The articulation assembly may also include a cone with spiral channels and/or pulleys to increase the distance the articulation member(s) 202 travels for a given linear length of the assembly. Combinations of the above are envisioned as well.

Although shown as including a single tensioning mechanism 208 in FIG. 1, it should be appreciated that the specific number of tensioning mechanisms 208 may be increased in alternate embodiments of the disclosure. For example, in embodiments of the endovascular system 10 that includes multiple articulation members 202, it is envisioned that each articulation member 202 may optionally be secured (connected) to a corresponding tensioning mechanism 208 (e.g., such that the number of articulation members 202 corresponds to the number of tensioning mechanisms 208). Embodiments in which a plurality of articulation members 202 may be secured (connected) to a single tensioning mechanism 208 are also envisioned herein, however, as are embodiments in which the tensioning mechanism(s) 208 may be omitted altogether. In those embodiments that are devoid of any tensioning mechanisms 208, it is envisioned that force may be manually, electronically, magnetically or otherwise applied to the articulation member(s) 202 in order to reconfigure the catheter 100.

With reference to FIG. 1, the stiffening assembly 300 includes respective proximal and distal ends 302, 304, and is configured for axial movement within the steerable segment 116 (e.g., in generally parallel relation to the longitudinal axis X (FIG. 1)) in order to vary the stiffness thereof, and thereby influence reconfiguration of the catheter 100. Stated differently, the steerable segment 116 includes a variable stiffness that can be altered, i.e., increased and decreased, via advancement and retraction of the stiffening assembly 300 in order to facilitate or restrict bending of the steerable segment 116 and select a length of curve zone and resulting radius bend for the catheter. Thus, the length of catheter 100 that will bend can be selectively decided by the clinician to provide a different turn radius (e.g., radius of curvature) and/or shape of turn. Thus, the catheter resists bending where the stiff piece is located.

In some embodiments, axial advancement (distal movement) of the stiffening assembly 300 through the steerable segment 116 which increases the stiffness thereof, can be used to inhibit (and in some embodiments entirely prevent) bending of a segment of the steerable segment 116, whereas axial retraction (proximal movement) of the stiffening assembly 300 through the steerable segment 116 could be used to decrease the stiffness thereof, which facilitates bending of a longer length of the steerable segment 116. Bending of a longer length will often result in a curve with a larger radius of curvature as well.

The stiffening assembly 300 includes a push member 306, e.g., a push rod or a push wire, (or alternatively a pull wire or pull rod; and or a dual function member) and a stiffening member 308 (e.g., a dowel 310 including a generally linear configuration) that is secured (connected) to the push member 306, preferably at a distal end thereof, such that movement of the push member 306 (e.g., axial advancement and retraction) causes corresponding movement (axial advancement and retraction) of the stiffening member 308. In the illustrated embodiment, the stiffening assembly 300 is unitary (e.g., monolithic) in construction, whereby the push member 306 and the stiffening member 308 are integrally formed from a single piece of material, either metallic (e.g., stainless steel, titanium, etc.) or non-metallic (e.g., plastic material(s), polymeric material(s), composite material(s)), or combination thereof. Various combinations of materials may be used as well. It is also envisioned, however, that the push member 306 and the stiffening member 308 may be configured as discrete components of the stiffening assembly 300, which may be secured (connected) together in any suitable manner such as, for example, via welding, via an adhesive, via (one or more) at least one mechanical fastener (e.g., pin(s), screw(s), clip(s), or the like), etc. Although shown as including a single push member 306 and a single stiffening member 308 in the illustrated embodiment, it should be appreciated that the specific number of push members 306 and stiffening members 308 may be increased in alternate embodiments of the disclosure. In such embodiments, it is envisioned that the push members 306 and the stiffening members 308 may act along the same length (e.g., segment) of the catheter 100 or along different lengths (e.g., segments) of the catheter 100 or can overlap along partial segments. They may act along lengths of the same and/or different steering segments.

In the illustrated embodiment, the push member 306 and the stiffening member 308 each include a generally annular transverse cross-sectional configuration. More specifically, the push member 306 defines a (first) transverse cross-sectional dimension (e.g., a diameter) D1 (FIG. 1), and the stiffening member 308 defines a (second) transverse cross-sectional dimension (e.g., a diameter) D2 that is larger than the transverse cross-sectional dimension D1. Embodiments are also envisioned, however, in which the push member 306 and/or the stiffening member 308 may include a generally non-annular transverse cross-sectional configuration (e.g., oval, square, rectangular, triangular, trapezoidal, diamond, pentagonal, hexagonal, heptagonal, octagonal, nonagonal, decagonal, etc.), as are embodiments in which the transverse cross-sectional dimensions D1, D2 respectively defined by the push member 306 and the stiffening member 308 may be substantially equivalent such that the stiffening assembly 300 includes a generally cylindrical (tubular) configuration that defines a generally uniform transverse cross-sectional dimension (e.g., a diameter) between the respective proximal and distal ends 302, 304 thereof. The stiffening member is preferably configured to remain substantially within the wall of the catheter. In embodiments wherein the stiffening member is cylindrical, it preferably remains substantially within the wall of the catheter along a circumference of the catheter, as a closed ring or partial ring.

The stiffening assembly 300 in some embodiments is spaced radially from the main lumen 106 and angularly (circumferentially) from the channel(s) 120. More specifically, the stiffening assembly 300 is some embodiments is within a passage 126 (FIG. 2) that is defined by the outer wall 104, and which is oriented in generally parallel relation to the longitudinal axis X (FIG. 1), the main lumen 106, and the channel(s) 120.

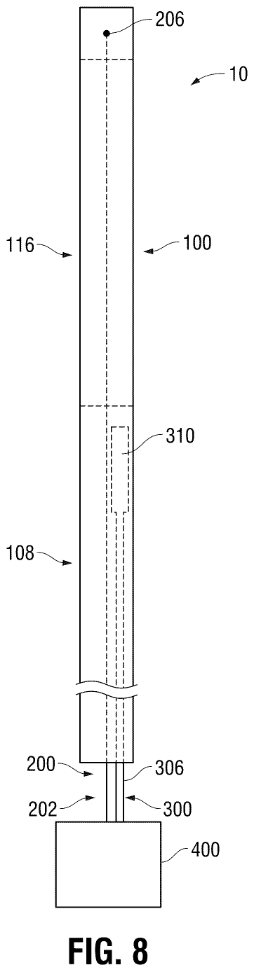

In some embodiments, the stiffening assembly 300 may be configured for manual manipulation (e.g., axial movement). Alternatively, the endovascular system 10 may include a controller 400 (FIG. 8) that is secured (connected) to the stiffening assembly 300 (e.g., the push member 306) such that axial advancement and retraction of the stiffening assembly 300 is regulated (governed) by the controller 400. In such embodiments, it is envisioned that the controller 400 may also be secured (connected) to the articulation assembly 200 such that that the application of force to the articulation member(s) 202 is also regulated (governed) by the controller 400. Multiple controllers are also contemplated. Additionally, a pull force instead of a push force for the stiffening assembly, or a combination thereof, is also contemplated.

The controller 400 may be positioned in any suitable location. For example, it is envisioned that the controller 400 may define, or may be included on, a handle of the catheter 100. In such embodiments, it is envisioned that the handle may include (optional) pulleys, spirals, levers, wheels, and/or other such mechanisms in order to increase the distance traveled by the articulation member(s) 202 and/or the push member 306 over a given distance, which allows an overall length of the handle to be reduced.

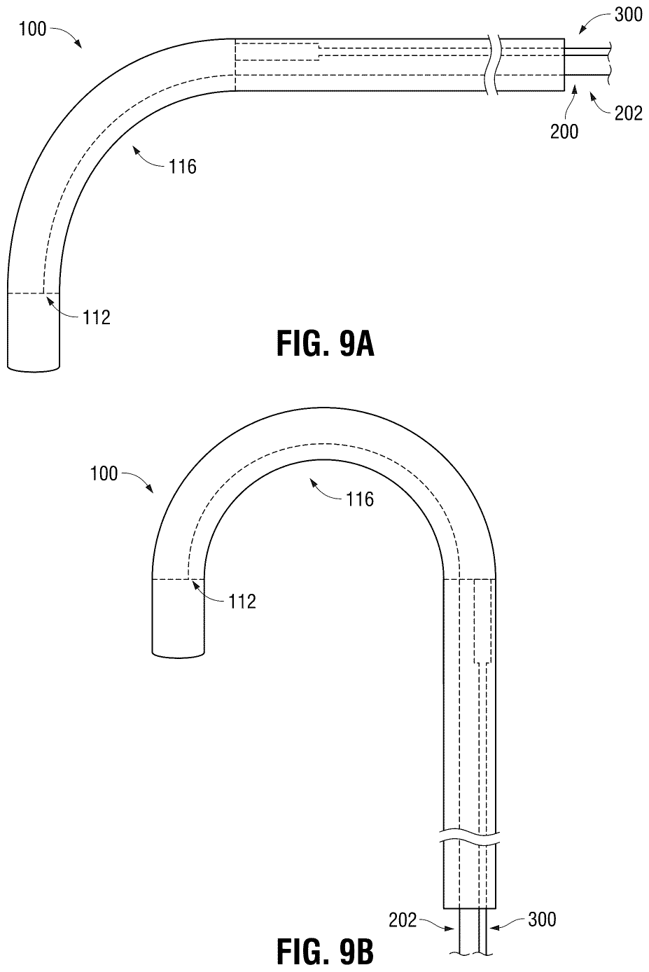

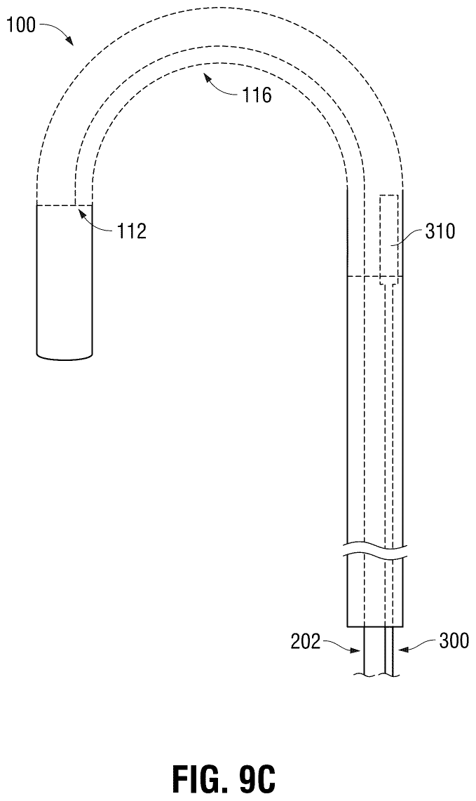

With reference now to FIGS. 1, 2, and 9A-11, a method of performing an endovascular procedure will be discussed using the endovascular system 10, additional details of which are provided in U.S. application Ser. No. 16/602,469, Publication No. 2020/0078554 and U.S. application Ser. No. 17/423,502, Publication No. 2022/0118219, the entire contents of both of which are hereby incorporated by reference. Initially, the catheter 100 is inserted into a patient's blood vessel with the catheter 100 in a (first, initial) normal configuration (FIG. 1), in which the catheter 100 is generally linear in configuration. The catheter 100 is then advanced through the blood vessel to a target site (e.g., a blockage), and, if necessary or desired, the articulation assembly 200 is actuated to reconfigure the catheter 100 from the initial configuration into a (second, subsequent) deflected configuration (FIG. 9A), in which the catheter 100 is generally non-linear (e.g., curved) in configuration. More specifically, upon actuation of the articulation assembly 200, force is applied to the catheter 100 (e.g., the distal segment 112) via the articulation member(s) 202, either manually or via the tensioning mechanism 208 (FIG. 1), which results in bending of the steerable segment 116.

Although the catheter 100 (e.g., the steerable segment 116) is illustrated as including a curvature of (approximately) 90 degrees in the deflected configuration in FIG. 9A, it should be appreciated that the curvature exhibited by the catheter 100 may be increased or decreased by adjusting the force that is applied thereto via the articulation assembly 200. For example, curvatures less than 90 degrees (e.g., approximately 15 degrees to approximately 75 degrees) and greater than 90 degrees (e.g., approximately 105 degrees to approximately 180 degrees or greater) are also envisioned herein, as seen in FIGS. 9B and 9C, for example, and would not be beyond the scope of the present disclosure. More specifically, FIG. 9B illustrates the catheter 100 in the deflected configuration with the steerable segment 116 curved by (approximately) 180 degrees and the stiffening assembly 300 removed (e.g., spaced proximally) therefrom. That is, the stiffening assembly is completely out of the “steer zone” (and showing an optional maximum 180 degree turn diameter). Similarly, FIG. 9C illustrates the catheter 100 in the deflected configuration with the steerable segment 116 curved by (approximately) 180 degrees. In contrast to the illustration provided in FIG. 9B, in which the steerable segment 116 defines a first radius of curvature, in FIG. 9C, the stiffening assembly 300 is partially advanced into the steerable segment 116 and the articulation member 202 is retracted a different amount (when compared to amount of retraction in FIG. 9B), which results in a second, reduced radius of curvature. Thus, as compared to FIG. 9B, in FIG. 9C the stiffener is partly into the steer zone and the pull mechanism is pulled (or push mechanism pushed) a different amount, but also pulled until the turn is approximately 180 degrees, resulting in a smaller “turn of diameter” curve. Curves of any degrees and/or shapes are also envisioned.

In some embodiments of the disclosure, it is envisioned that the catheter 100 may include a locking mechanism that can be manually or automatically actuated (e.g., via the controller 400 (FIG. 8)) in order to fix the curvature of the catheter 100 in a desired deflected configuration.

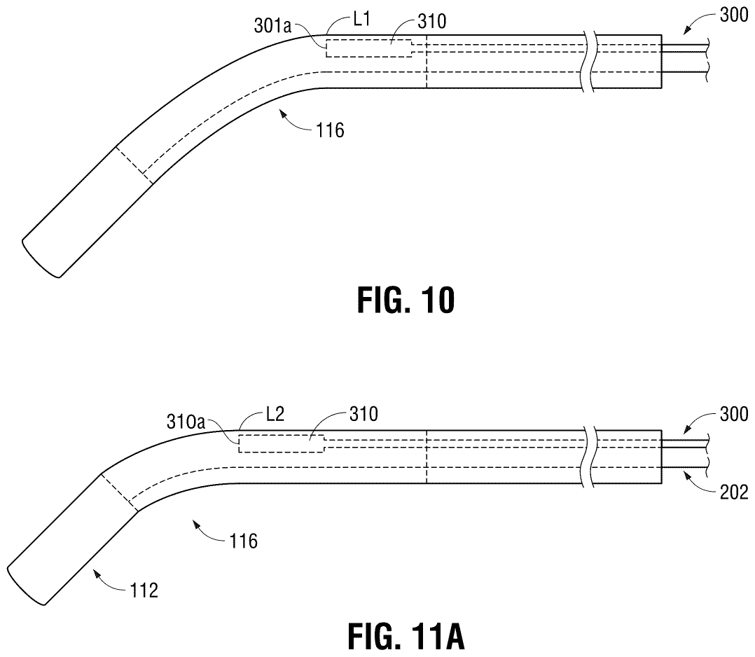

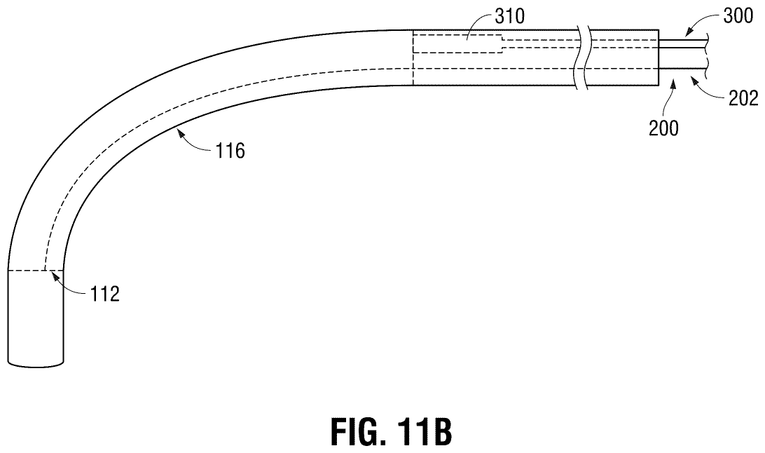

Depending upon the particular procedure being performed, the location of the pathology, etc., following reconfiguration of the catheter 100, it may be necessary or desirable to alter the configuration thereof by reducing the curvature in the deflected configuration via manipulation of the stiffening assembly 300. More specifically, advancing the stiffening assembly 300 axially within the passage 126 (FIG. 2) increases the stiffness of the steerable segment 116, which reduces bending of the catheter 100 (e.g., the length of catheter being bent, and/or radius of curvature), and allows for steering of the distal segment 112 (e.g., the distal end hole 114 and/or the side hole(s) 118 (FIG. 3)) to increase not only control over the specific position of the catheter 100, but precision in the placement thereof. For example, FIG. 10 illustrates advancing the stiffening assembly 300 within the steerable segment 116 to a (first) axial location L1 (reference numeral 310a denoting the distalmost end of the stiffening member 310) so as to reduce the curvature of the catheter 100 to (approximately) 45 degrees in the deflected configuration (and decrease the curve zone), and FIG. 11A illustrates advancing the stiffening assembly 300 within the steerable segment 116 to a (second) axial location L2 so as to reduce the curvature of the catheter 100 to (approximately) 15 degrees in the deflected configuration (and further decrease the curve zone). FIG. 11B illustrates positioning of the stiffening member 310 of stiffening assembly 300 within the steerable segment 116 to a (third) axial location to achieve a greater curvature. Note these various positions of the stiffening member are shown in the drawings by way of example, as other positions to achieve other curvatures are also contemplated. That is, it is envisioned that any desirable curvature for the catheter 100 may be realized via manipulation of the articulation assembly 200 and the stiffening assembly 300 (e.g., from (approximately) 180 degrees to (approximately) 0 degrees). Thus, the bend diameter/turning radius can be altered (increased or decreased) via movement of the stiffening member into variable lengths of a given “steer” zone to effectively further stiffen a segment it is moved into to resist bending. The stiffener is optimally positioned before bending the catheter, but alternative methods such as that described above wherein the stiffener is positioned after bending the catheter are also envisioned.

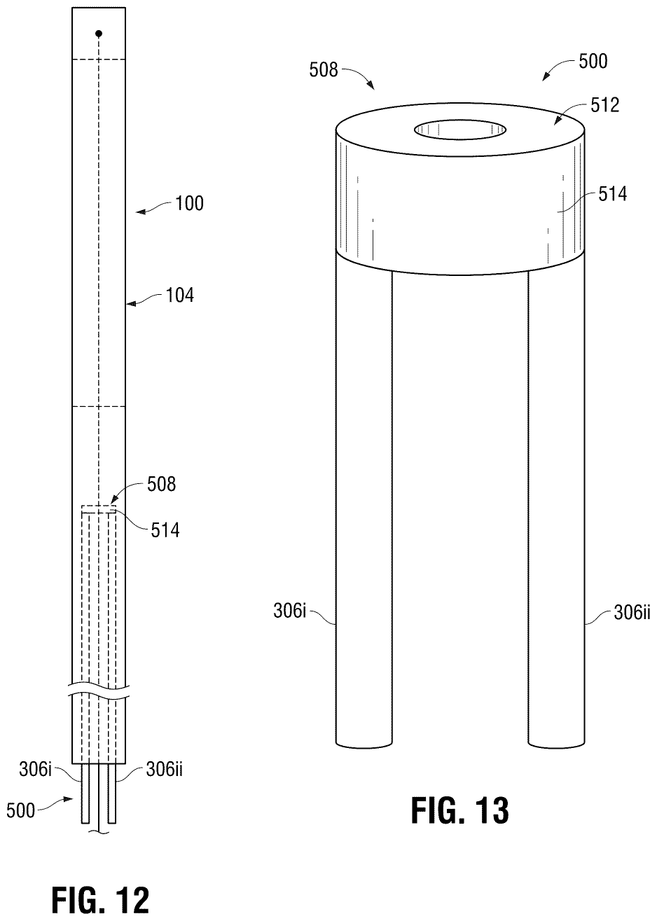

With reference now to FIGS. 12 and 13, an alternate embodiment of the stiffening assembly 300 will be discussed, which is identified by the reference numeral 500. The stiffening assembly 500 is substantially similar in both structure and function to the aforedescribed stiffening assembly 300 (FIG. 1) and, accordingly, will only be discussed with respect to differences therefrom in the interest of brevity. As such, identical reference numerals will be utilized to refer to elements, structures, features, etc., common to the stiffening assemblies 300, 500.

The stiffening assembly 500 includes a pair of (first and second) elongated members such as push members 306i, 306ii, and an alternate embodiment of the stiffening member 308, which is identified by the reference numeral 508. In contrast to the stiffening member 308, the stiffening member 508 includes a generally non-linear configuration. More specifically, the stiffening member 508 includes an arcuate (curved) configuration, and is configured as a stiffening ring 512 having a generally toroidal configuration. An embodiment in which the stiffening member 508 may be crescent shaped (e.g., semicircular) in configuration, cylindrical, or other configurations is also envisioned herein.

Although shown as including a pair of push members 306 in the embodiment illustrated in FIGS. 12 and 13 that extend proximally from the stiffening member 508, embodiments in which the stiffening assembly 500 may include a single push member 306 are also envisioned herein.

Although generally described as push members 306, it should be appreciated that the stiffening assembly 500 may also be as a pull member or configured for pulling as well. Any push or pull member may alternatively be configured to both push and/or pull, as desired by the operator. Additionally, it is envisioned that one or more wires and/or other such elongated control members may be incorporated as well.

As discussed above in connection with the stiffening assembly 300 (FIG. 1), the stiffening assembly 500 may be configured for manual manipulation, or that the stiffening assembly 500 (e.g., the push member(s) 306) may be secured (connected) to the controller 400 (FIG. 8) such that axial advancement and retraction of the stiffening assembly 500 is regulated (governed) by the controller 400.

In the illustrated embodiment, the stiffening ring 512 includes a hollow construction substantially cylindrical in configuration and is configured as a hypotube 514 that defines an internal lumen. Embodiments in which the configuration of the stiffening ring/hypotube 512 may be varied are also envisioned. For example, embodiments in which the stiffening ring 512 may include a solid construction (e.g., embodiments in which the stiffening ring 512 is devoid of the internal lumen) are also envisioned herein, as are embodiments in which the stiffening ring 512 may include (one or more) at least one weakened section (e.g., etching, laser cuts, etc.) to increase the flexibility thereof. In such embodiments, the stiffening ring 512 may include a stiffness that is greater than that of the steerable segment 116, but less than that of the proximal segment 108. It may also facilitate alternate shapes of bending. Additionally, or alternatively, it is envisioned that the catheter 100 (e.g., the outer wall 104) may include (one or more) at least one weakened section (e.g., etching, laser cuts, etc.) in certain embodiments in order to facilitate deflection of the catheter 100 so as to achieve a curvature (e.g., bends) with a particular configuration and/or dimensions. The hypotube can optionally slide over or under a laser cut hypotube, when the laser cut hypotube is incorporated to help influence the bend segment and shape. That is, in some embodiments, the steerable segment can include at least one fixed hypotube embedded in the catheter wall with a first laser cut pattern to facilitate bending in a first predetermined shape, wherein upon activation of a steering force, the steerable segment assumes at least one predetermined configuration. The hypotube in some embodiments can further have a second cut pattern to facilitate bending in a second predetermined shape so the steerable segment assumes a different predetermined configuration. The first and second patterns can be on opposite sides in some embodiments.

While the stiffening member 508 is shown as being located internally within the catheter 100 (e.g., as extending within or substantially within the outer wall 104), embodiments are also envisioned in which the stiffening member 508 may be located fully externally or partially externally of the catheter 100. For example, embodiments in which the stiffening member 508 may (partially or entirely) circumscribe the outer wall 104 of the catheter 100 are also envisioned herein, as are embodiments in which the stiffening member 508 may be positioned entirely within the outer wall 104, and embodiments in which the stiffening member 508 may be positioned partially within the outer wall 104 and partially exposed from the outer wall 104.

The stiffening ring 512 can be of different lengths than that shown. Additionally, it is envisioned that the stiffening ring 512 may be configured as an elongated hypotube or rod of a different length than shown in FIG. 13. Additionally, ring 512 may have one or more push/pull members (although 2 are shown in FIG. 13.

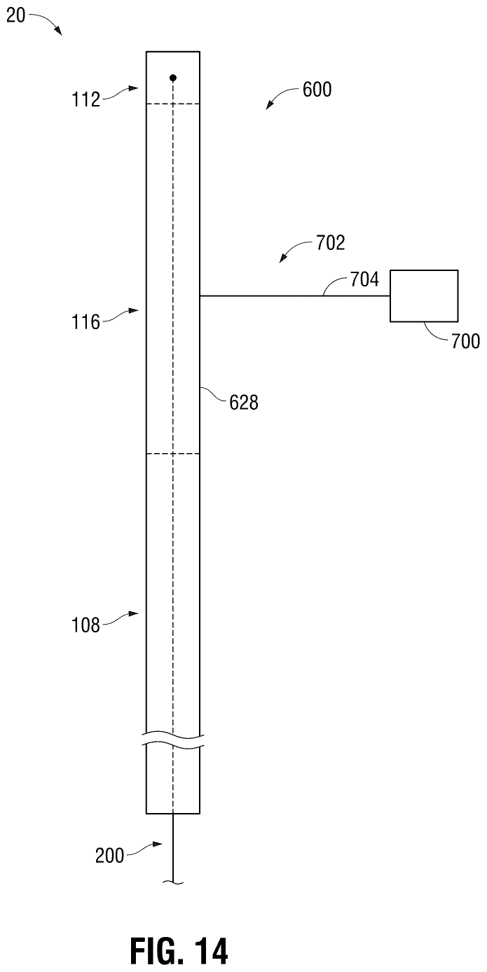

With reference now to FIG. 14, an alternate embodiment of the endovascular system 10 will be discussed, which is identified by the reference numeral 20, and includes a catheter 600 and the articulation assembly 200. The endovascular system 20 and the catheter 600 are substantially similar in both structure and function to the endovascular system 10 and the catheter 100 discussed above (FIG. 1) and, accordingly, will only be discussed with respect to differences therefrom in the interest of brevity. As such, identical reference numerals will be utilized to refer to elements, structures, features, etc., common to the endovascular systems 10, 20 and the catheters 100, 600. In contrast to the endovascular system 10, in which stiffness in the steerable segment 116 is varied mechanically via manipulation of the stiffening assembly 300, the endovascular system 20 is devoid of the stiffening assembly 300, and instead utilizes (one or more) at least one shape memory material 628 that is responsive to an external stimulus in order to vary the stiffness of the steerable segment 116. More specifically, the shape memory material(s) are incorporated into the steerable segment 116 such that, upon exposure to the external stimulus, the stiffness of the steerable segment 116 is altered, i.e., increased or decreased. The catheter 600 thus includes a first material of construction in the proximal segment 108, and a second, different material of construction (e.g., the shape memory material(s)) in the steerable segment 116. As discussed above in connection with the catheter 100 (FIG. 1), the material of construction in the distal segment 112 may be the same or different than that in the proximal segment 108. Alternatively, the stiffness of the section may be altered by having pockets within it to accept additions and subtractions of additional substances that may alter the stiffness of a part of the steer zone.

In certain embodiments, it is envisioned that the shape memory material(s) may be incorporated into the catheter 100 such that, upon exposure to the external stimulus, the steerable segment 116 assumed a (predetermined) configuration. For example, it is envisioned that the external stimulus may cause the steerable segment 116 to assume a bend with a certain length and/or radius of curvature.

In the illustrated embodiment, the catheter 600 is configured such that the shape memory material(s) are responsive to an electrical stimulus (e.g., electrical current), which is communicated to the steerable segment 116 from a power source 700 via (one or more) at least one transmission member 702 (e.g., a wire 704). Embodiments in which the shape memory material(s) may be responsive to a heat stimulus (e.g., intracorporeal heat from the patient) are also envisioned herein, however, which would allow for omission of the power source 700 and the transmission member(s) 702. Embodiments in which the catheter 600 may include and/or may be configured for use with robotic and/or automated components are envisioned as well.

Depending upon the particular procedure in which the endovascular system 20 is employed, and the particular memory material(s) used in construction of the catheter 600, the catheter 600 may be configured such that the stiffness of the steerable segment 116 is increased upon exposure to the external stimulus to thereby decrease the curvature of the catheter 600 in the deflected configuration (FIGS. 9A-11). Alternatively, the catheter 600 may be configured such that the stiffness of the steerable segment 116 is decreased upon exposure to the external stimulus to thereby increase the curvature of the catheter 600 in the deflected configuration. The catheter 600 thus allows an operating clinician to selectively stiffen and/or soften various regions of the catheter 600 as desired. Transmission members can be provided to communicate with different regions of steerable segment 116 to selectively stiffen select regions of the steerable segment to create various regions of bend resistance. The multiple transmission members can be selectively powered by a single power source 700 or by individual power sources for one or more transmission members.

With continued reference to FIG. 14, a method of performing an endovascular procedure will be discussed using the endovascular system 20. Following insertion of the catheter 600 into the patient's blood vessel (e.g., with the catheter 600 in the normal configuration (FIG. 14)), the catheter 600 is advanced to the target site, during or after which, the articulation assembly 200 can be actuated in order to reconfigure the catheter 600 via bending of the steerable segment 116. If necessary or desired, during the course of the endovascular procedure, the configuration of the catheter 600 can be altered by varying the stiffness of the steerable segment 116 via exposure to the external stimulus. More specifically, the catheter 600 can be exposed to the external stimulus to thereby increase the stiffness of the steerable segment 116 and, thus, reduce bending of the catheter 600 (e.g., the radius of curvature).

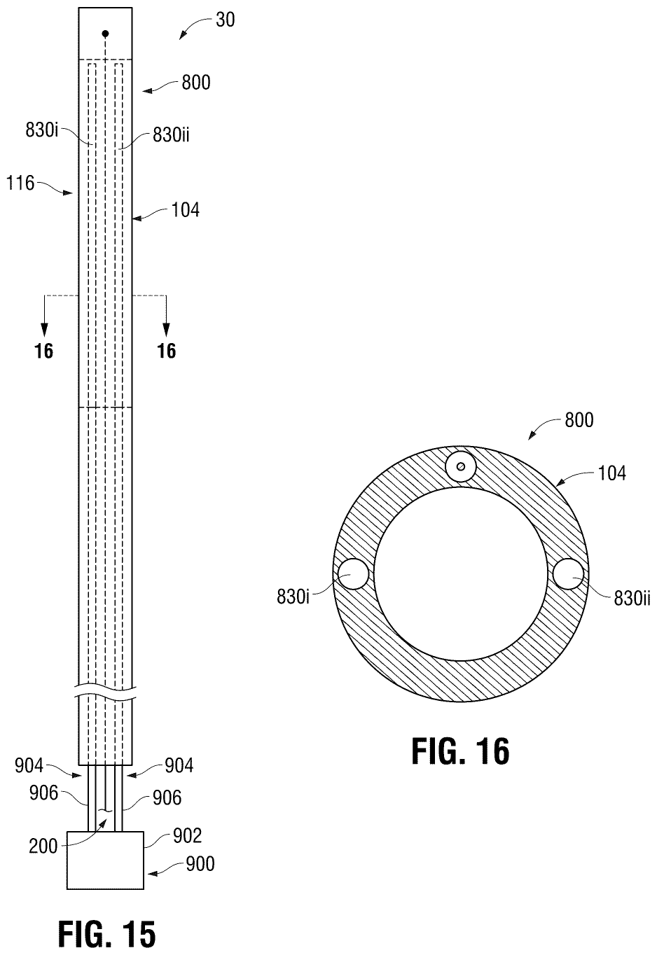

With reference now to FIGS. 15 and 16, an alternate embodiment of the endovascular system 10 will be discussed, which is identified by the reference numeral 30, and includes a catheter 800 and the articulation assembly 200. The endovascular system 30 and the catheter 800 are substantially similar in both structure and function to the endovascular system 10 and the catheter 100 discussed above (FIG. 1) and, accordingly, will only be discussed with respect to differences therefrom in the interest of brevity. As such, identical reference numerals will be utilized to refer to elements, structures, features, etc., common to the endovascular systems 10, 30 and the catheters 100, 800.

The catheter 800 includes (one or more) at least one (internal) chamber 830 (e.g., a cavity, a pocket, etc.) that is defined by the outer wall 104. The chamber(s) 830 are located within the steerable segment 116 and are configured to receive a fluid (e.g., air, water, saline, or other such suitable substance) from a source 900 (e.g., a fluid pump 902), which allows the stiffness of the steerable segment 116 to be varied. More specifically, communication of the fluid (substance) into the chambers(s) 830 (e.g., from the source 900) causes the stiffness of the steerable segment 116 to be increased, whereas communication of the fluid (substance) out from the chamber(s) 830 (e.g., to the source 900) causes the stiffness of the steerable segment 116 to be decreased. The extent of filling the chamber can be utilized to adjust the catheter bend by selecting the desired stiffer segment based on where the stiffening fluid (substance) ends in the catheter.

In various embodiments, it is envisioned that a single chamber 830 may be utilized to increase the stiffness of the catheter 800 when filled with a certain substance or decrease the stiffness of the catheter 800 if the substance is removed.

It is also envisioned that a single chamber 830 may be utilized to decrease the stiffness of the catheter 800 when filled with a certain substance or increase the stiffness of the catheter 800 if the substance is removed.

Embodiments in which the stiffness of the catheter 800 may be varied by adding and/or removing different substances are also envisioned herein.

Although shown as including a pair of (first and second) chambers 830i, 830ii that are positioned in generally diametrical opposition, it should be appreciated that the particular number of chambers 830 may be increased or decreased in alternate embodiments without departing from the scope of the present disclosure. For example, embodiments are envisioned in which the catheter 800 may include a single chamber 830, as are embodiments in which the catheter 800 may include three or more chambers 830. Chambers of various shapes, number and position are envisioned, including cylindrical chambers in the circumference of the wall of the device. By filling different chambers, the catheter stiffness can be varied, i.e., the clinician can decide which segment of the catheter to stiffen to adjust its bend, and/or the stiffness of different regions of the catheter 800 may be altered. The multiple chambers can be placed circumferentially aligned and/or axially spaced and can simultaneously or selectively receive fluid.

The chamber(s) 830 are in communication with the fluid (liquid or gas) source 900 via (one or more) at least one conduit 904 (e.g., a tube 906) that is configured for connection to the catheter 800. For example, it is envisioned that the conduit(s) 904 may extend within or substantially within the outer wall 104 of the catheter 800 from a site near a proximal end thereof. The fluid (substance) may be communicated from the source 900 and into the chamber(s) 830 in any suitable manner such as, for example, through (one or more) at least one port, opening, seal, or the like in the body 102 of the catheter 800. A fluid pump(s), injection device, or other devices/mechanisms can be utilized to advance the fluid into the chamber(s) and a suction device(s) or other device/mechanism to remove the fluid can be utilized to withdraw the liquid from the chamber(s). With continued reference to FIGS. 15 and 16, a method of performing an endovascular procedure will be discussed using the endovascular system 30. Following insertion of the catheter 800 into the patient's blood vessel (e.g., with the catheter 800 in the normal configuration (FIG. 15)), the catheter 800 is advanced to the target site, during or after which, the articulation assembly 200 can be actuated in order to reconfigure the catheter 800 via bending of the steerable segment 116. If necessary or desired, during the course of the endovascular procedure, prior or subsequent to insertion of the catheter into the body, the configuration of the catheter 800 can be altered by varying the stiffness of the steerable segment 116 via communication of the fluid (substance) into the chamber(s) 830 (e.g., from the 900) and out of the chamber(s) 830 (e.g., to the source 900). More specifically, communication of the fluid (substance) into the chamber(s) 830 increases the stiffness of the steerable segment 116, thereby reducing bending of the catheter 800 (e.g., the and/or the length of the steerable segment 116 that will bend), and communication of the fluid (substance) from the chamber(s) 830 (e.g., to the source 900) decreases the stiffness of the steerable segment 116, thereby facilitating bending of the catheter 800 (e.g., an increase in the radius of curvature). As such, by varying the volume of the fluid (substance) within the chamber(s) 830, the stiffness of the steerable segment 116 and, thus, the length of curve and/or the curvature of the catheter 800 in the deflected configuration, can be controlled (regulated) with increased precision.

In those embodiments of the catheter 800 including a plurality of chambers 830, chambers 830 may be in communication with each other so as to permit communication of the fluid (substance) into the chambers 830 from the source 900 via a single conduit 904, which results in uniform stiffness in the steerable segment 116. Alternatively, the chambers 830 may be devoid of communication, (i.e., independent of each other) which allows the chambers 830 to be filled independently from the source 900 (or multiple sources 900) via corresponding conduits 904, and results in non-uniform stiffness in the steerable segment 116 and/or selective segment stiffening. For example, in such embodiments, and the chamber 830i may receive a (first) volume of the fluid (substance), and the chamber 830ii may receive a (second) volume of the fluid (substance), which may be less than, equal to, or greater than the first volume of the fluid (substance), thereby facilitating additional control over the configuration of the catheter 800. Axially aligned chambers can enable selection via filling of the axial location of the stiffening to select the desired bend radius or curve zone.

In preferred embodiments, the stiffening assemblies disclosed herein will minimally affect the amount of degrees of curvature, although it can sometimes do so to a somewhat unpredictable degree. The stiffening assembly primary purpose is to reduce the length of the catheter that will bend. By reducing the length of catheter bending, many configurations will result in a reduced radius of the curve that the steer will create.

The steerable system is described above for steering a catheter in endovascular procedures. However, it should be appreciated that the steerable systems of the present invention to adjust stiffness can be used in catheters for other procedures as well as used to steer endoscopes for various applications, e.g., gastrointestinal, genito-urinary, pulmonary, enterology, etc. In some of these applications/embodiments, an incorporated imaging system, e.g., camera, and/or lighting system, may be included in the catheter (or endoscope) as well.

The catheter in preferred embodiments has one central working lumen, but in some embodiments can have multiple “working” lumens in addition to the lumens in the wall. One of the lumens can be used for an imaging system.

The steerability system is described above as manually activated, however, it should be appreciated that it could alternatively be robotically activated, computer driven, hydraulically driven, motor driven, etc. Automated artificial intelligence driven steering is envisioned as well.

Steerability systems such as described in U.S. application Ser. No. 18/668,492, filed May 20, 2024, the entire contents of which are incorporated herein by reference, can also be utilized. Although the apparatus and methods of the subject disclosure have been described with respect to preferred embodiments, those skilled in the art will readily appreciate that changes and modifications may be made thereto without departing from the spirit and scope of the present disclosure as defined by the appended claims.

Persons skilled in the art will understand that the various embodiments of the disclosure described herein and shown in the accompanying figures constitute non-limiting examples. Additionally, persons skilled in the art will understand that the elements and features shown or described in connection with one embodiment may be combined with those of another embodiment without departing from the scope of the present disclosure and will appreciate further features and advantages of the presently disclosed subject matter based on the description provided.

In the preceding description, reference may be made to the spatial relationship between the various structures illustrated in the accompanying drawings, and to the spatial orientation of the structures. However, as will be recognized by those skilled in the art after a complete reading of this disclosure, the structures described herein may be positioned and oriented in any manner suitable for their intended purpose. Thus, the use of terms such as “above,” “below,” “upper,” “lower,” “inner,” “outer,” “left,” “right,” “upward,” “downward,” “inward,” “outward,” etc., should be understood to describe a relative relationship between the structures and/or a spatial orientation of the structures. Those skilled in the art will also recognize that the use of such terms may be provided in the context of the illustrations provided by the corresponding figure(s).

Additionally, terms such as “approximately,” “generally,” “substantially,” and the like should be understood to allow for variations in any numerical range or concept with which they are associated and encompass variations on the order of 25% or to allow for manufacturing tolerances and/or deviations in design.

Although terms such as “first,” “second,” “third,” etc., may be used herein to describe various operations, elements, components, regions, and/or sections, these operations, elements, components, regions, and/or sections should not be limited by the use of these terms in that these terms are used to distinguish one operation, element, component, region, or section from another. Thus, unless expressly stated otherwise, a first operation, element, component, region, or section could be termed a second operation, element, component, region, or section without departing from the scope of the present disclosure.

Each and every claim is incorporated as further disclosure into the specification and represents embodiments of the present disclosure. Also, the phrases “at least one of A, B, and C” and “A and/or B and/or C” should each be interpreted to include only A, only B, only C, or any combination of A, B, and C.

1. A system configured for use during an endovascular procedure, the system comprising:
a catheter configured for insertion into a patient's blood vessel, the catheter including:
a proximal segment; and
at least one steerable segment located distal to the proximal segment;
a steering assembly operatively connected to the catheter and configured to facilitate reconfiguration thereof, wherein the proximal segment has a first stiffness, and the steerable segment has a second stiffness less than the first stiffness to facilitate bending of the steerable segment during reconfiguration of the catheter; and
a stiffening assembly configured for movement with respect to the steerable segment to increase the second stiffness and restrict bending thereof.

2. The system of claim 1, wherein the steering assembly includes:
at least one steering member extending within the catheter and secured thereto; and
a tensioning mechanism connected to the at least one steering member such that upon actuation of the tensioning mechanism, a force is applied to the catheter via the at least one steering member to facilitate reconfiguration thereof.

3. The system of claim 2, wherein the at least one steering member extends substantially within an outer wall of the catheter.

4. The system of claim 2, wherein the at least one steering member extends within a channel defined by the outer wall of the catheter.

5. The system of claim 2, wherein the at least one steering member includes:
a first steering member secured to the catheter such that upon actuation of the tensioning mechanism, the first steering member causes deflection of the catheter in a first direction; and
a second steering member secured to the catheter such that, upon actuation of the tensioning mechanism, the second steering member causes deflection of the catheter in a second direction.

6. The system of claim 1, wherein the catheter includes a distal segment distal of the steering segment and wherein the distal segment has a third stiffness greater than the second stiffness.

7. (canceled)

8. The system of claim 1, wherein the proximal segment includes a first material having a first durometer, and the steerable segment includes a second material having a second durometer, wherein the second durometer is less than the first durometer.

9. (canceled)

10. The system of claim 1, wherein the stiffening assembly includes:
a push member; and
a stiffening member secured to the push member such that movement of the push member causes corresponding movement of the stiffening member.

11. The system of claim 1, wherein the steering assembly includes a steering member, and further comprising a controller connected to the steering member and configured to cause axial movement of the steering member to cause axial movement of the stiffening assembly within the steerable segment.

12. The system of claim 1, wherein the stiffening assembly includes at least one hypotube substantially within a wall of the catheter and movable within the wall.

13. The system of claim 1, wherein the stiffening assembly includes at least one substantially cylindrical member substantially within a wall of the catheter and movable within the wall.

14. The system of claim 1, wherein moving the stiffening assembly alters the length of the steering segment, wherein when the steering segment is shortened by positioning of the stiffening assembly, the turning radius is decreased.

15. (canceled)

16. (canceled)

17. The system of claim 1, wherein the steerable segment includes at least one shape memory material such that, upon exposure to an external stimulus, the steerable segment assumes a predetermined configuration.

18. (canceled)

19. The system of claim 1, wherein the steerable segment includes at least one fixed hypotube embedded in the catheter wall with a first cut pattern to facilitate bending in a first predetermined shape, wherein upon activation of a steering force, the steerable segment assumes at least one predetermined configuration.

20. The system of claim 19, wherein the hypotube has a second cut pattern to facilitate bending in a second predetermined shape so the steerable segment assumes a different predetermined configuration.

21-34. (canceled)

35. A method of performing an endovascular procedure, the method comprising:
a) inserting a catheter into a blood vessel in a first configuration, wherein the catheter includes:
a first segment; and
a second segment located distally of the first segment;
b) advancing the catheter towards a target site;
c) actuating a steering assembly connected to the catheter to thereby reconfigure the catheter from the first configuration into a second configuration via bending of the second segment; and
d) either prior to or subsequent to step (c), altering a stiffness of the second segment to alter bending thereof.

36. The method of claim 35, wherein increasing the stiffness of the second segment includes advancing a stiffening assembly axially within the second segment.

37. The method of claim 35, wherein increasing the stiffness includes moving a substantially cylindrical member positioned substantially in the wall of a catheter into a select region of the second segment via an elongated member attached thereto.

38. The method of claim 35, wherein increasing the stiffness of the second segment includes exposing the second segment to an external stimulus.

39. The method of claim 35, wherein increasing the stiffness of the second segment includes communicating a substance into at least one chamber located within the second segment.
